(19) United States
(12) Patent Application Publication
(10) Pub. No.: US 2004/0111069 A1
(43) Pub. Date: Jun. 10, 2004

(54) Connecting piece for a double lumen tube with two individual lumen tubes

(76) Inventors: Gunter Schaaf, Montevideo (UY); Karl Heniz Rittersberger, Heppenheim (DE)

Correspondence Address: Norris McLaughlin & Marcus, 30th Floor, 220 East 42nd Street, New York, NY, 10017, US

(21) Appl. No.: 10/469,908

(22) PCT Filed: Feb. 15, 2002
(86) PCT No.: PCT/EP02/01608

(30) Foreign Application Priority Data
Mar. 7, 2001 (UY) 26.609

(51) Int. Cl. A61M 25/00

(52) U.S. Cl. 604/284

(57) ABSTRACT

Connecting piece (16) for a double-lumen tube (12) with two single-lumen tubes (14; 16), particularly for use in the tracheobronchial system.

The connecting piece (26) has an input connection (28) for the double-lumen tube (12) branching into two output connections (30; 32) for the single-lumen tubes (14; 16), and the clear passages for the double-lumen tube (12) on the one hand and the output connections for the single-lumen tubes (14; 16) on the other hand are constructed in such a way that in their cross-sectional shape they have a substantially step-free continuous transition into the lumina of the tubes to be attached.

The invention relates to a connecting piece for a double-lumen tube with two single-lumen tubes, particularly for use in the tracheobronchial system.

Such tubes or catheters, also known as endobronchial tubes, in which at one end of a double-lumen tube at least two single-lumen tubes are each attached to one of the lumina of the double-lumen tube, are used for example as probes or catheters to be inserted for example into the trachea and/or one of the bronchi and serve for example for unilateral and/or bilateral respiration (ventilation), for draining of the tracheobronchial system or for introduction of gaseous or thin fluid media which are pharmaceutically active or which serve for diagnosis or as anaesthetic. Such endobronchial tubes, which may be equipped with an inflatable balloon for sealing off the trachea during use in intubation anaesthesia, are also used for the introduction of flexible fibre-optic bronchoscopes for controlling the position of the double-lumen tubes, for diagnostic purposes or for operative interventions. One of the single-lumen tubes which are each attached to a lumen of the double-lumen tube is also introduced into the trachea and the second one is introduced further into the right or left main bronchus, whereby the inflatable balloon provided in each of the separate lumina can close off the lumen or make it passable according to the requirements.

One problem with such tubes is the transition or branching region between the double-lumen tube and the single-lumen tubes. These are attached by means of connecting tubes pushed into the respective adjoining lumen at the associated end of the double-lumen tube or of the single-lumen tube, which leads to a step-like narrowing of the cross-section within the lumen. This leads to turbulence in the flowing air or gases and impedes the introduction and the movement of bronchoscopes and leads to damage to the sensitive outer skin of the movable part of the fibre optics, which can lead to destruction of the expensive instrument. In the best case an instrument damaged in this way can be sent away for cost-intensive repair. However, fibre-optic bronchoscopy is a diagnostic and treatment technique which is increasing in importance. Accordingly the object of the invention is to improve the known endotracheal tubes made up of two single-lumen tubes attached to one end of a double-lumen tube in such a way that the observed disadvantages in the transition or branching region are reliably avoided and thus there are no restrictions to the through flow or passage nor can there be any damage to bronchoscopes to be introduced into the tracheobronchial system.

This object is achieved according to the invention by a connecting piece for a double-lumen tube with two single-lumen tubes which has an input connection for the double-lumen tube branching into two output connections for the single-lumen tubes, the clear passages for the double-lumen tube on the one hand and the output connections for the single-lumen tubes on the other hand being constructed in such a way that in their cross-sectional shape they ensure a substantially step-free continuous transition into the lumina of the tubes to be attached.

In a preferred embodiment of the invention the connecting piece has the shape of a Y in which a lumen section extending within the input connection for the double-lumen tube branches into two output lumen sections coordinated with the output connections for the single-lumen tubes.

In this case it is advantageous if the clear internal cross-section of the input connection intended for connection to the double-lumen tube is of substantially complementary shape to the outer wall of the double-lumen tube in the region where it is attached to the connecting piece. The internal cross-section of the input connection can then be provided in the region, opposite the end edge of the double-lumen tube in the proper connection position, of a narrowing of the cross-section forming a circumferential step abutment which is constructed in such a way that the remaining passage cross-section corresponds exactly to the passage cross-section of the double-lumen tube. When, during installation of the single-lumen tubes, the double-lumen tube is pushed as far as the circumferential step abutment, an automatic alignment and centring then takes place in such a way that there are no step-like transitions between the cross-sections of the lumina in the double-lumen tube and the passages in the output connections for the single-lumen tubes.

When the internal passage cross-section of the double-lumen tube is divided in a manner which is known per se by a continuous central web into two lumina which are approximately semicircular in cross-section, it is recommended that in the transition region between the connection for the double-lumen tube and the connections for the single-lumen tubes a slot-like recess be provided in the branching region, whereby in the proper connection position an extended portion of the web of the double-lumen tube then engages and fits in the said recess.

Also the output connections for the single-lumen tubes preferably have in the region where they are attached to the tubes a passage cross-section which is complementary to the outer wall of the single-lumen tubes in the connection region. The installation of the single-lumen tubes in the connecting piece then again takes place by insertion into the respective associated output connection, the said output connections again advantageously each having a circumferential step-like abutment to limit the depth of insertion of the single-lumen tubes, and again the clear passage cross-section of the connecting piece in the abutment region corresponds substantially to the cross-section of the associated lumen of the single-lumen tube to be attached.

The connecting piece is preferably made from a suitable thermoplastic plastics material which thus makes manufacture possible using the injection-moulding process. In this case a plastics material with elastomeric properties can also be used.

Alternatively, manufacture of the connecting piece from metal is also possible.

It may also be advantageous if the single-lumen tubes at the out put end are produced as an integral die-cast or injection-moulded part together with the connecting piece.

The ultimate tension-proof and compression-resistant sealed connection of the tubes to be attached to the connection piece can also be ensured if the regions where the connecting piece is attached to the tubes which are to be attached are constructed as a glued connection.

Alternatively, the connection of the connecting piece to the tubes to be joined on may also take place by injection-moulding the connecting piece directly onto the end portions of the tubes to be attached.

The invention is explained in greater detail in the following description in connection with the drawings, in which:

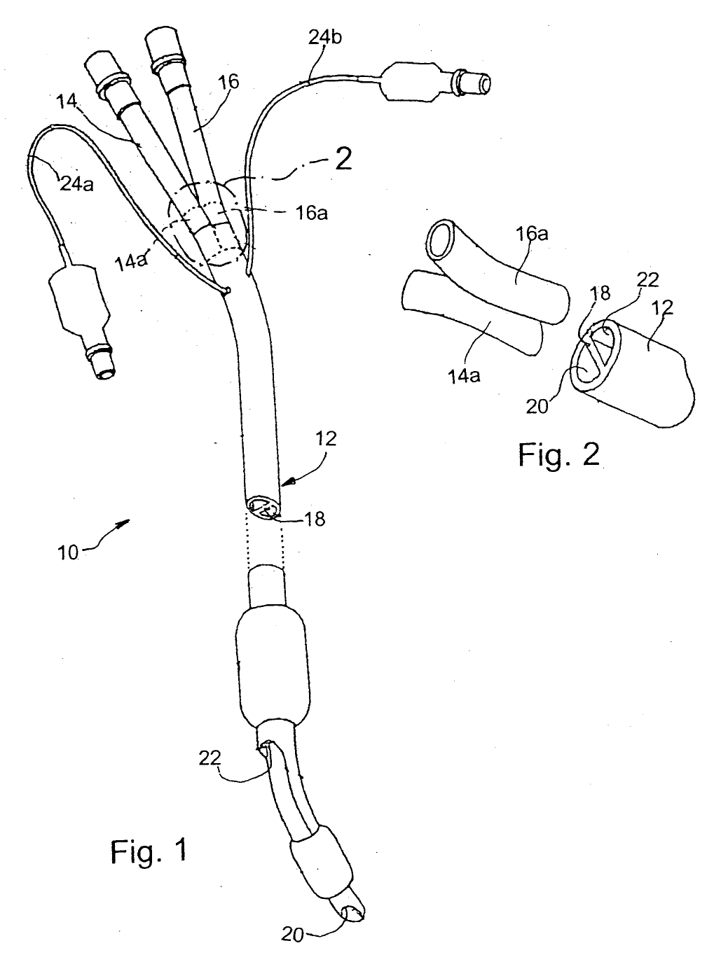

FIG. 1 shows a three-dimensional representation of a known form of the basic construction of an endotracheal tube made up of a double-lumen tube with two single-lumen tubes attached at one end;

FIG. 2 shows an exploded perspective representation of the end region of a double-lumen tube lying within the region enclosed by chain-dotted lines and the appertaining connecting tubes of the known endotracheal tube provided for connection to the single-lumen tubes which are to be attached;

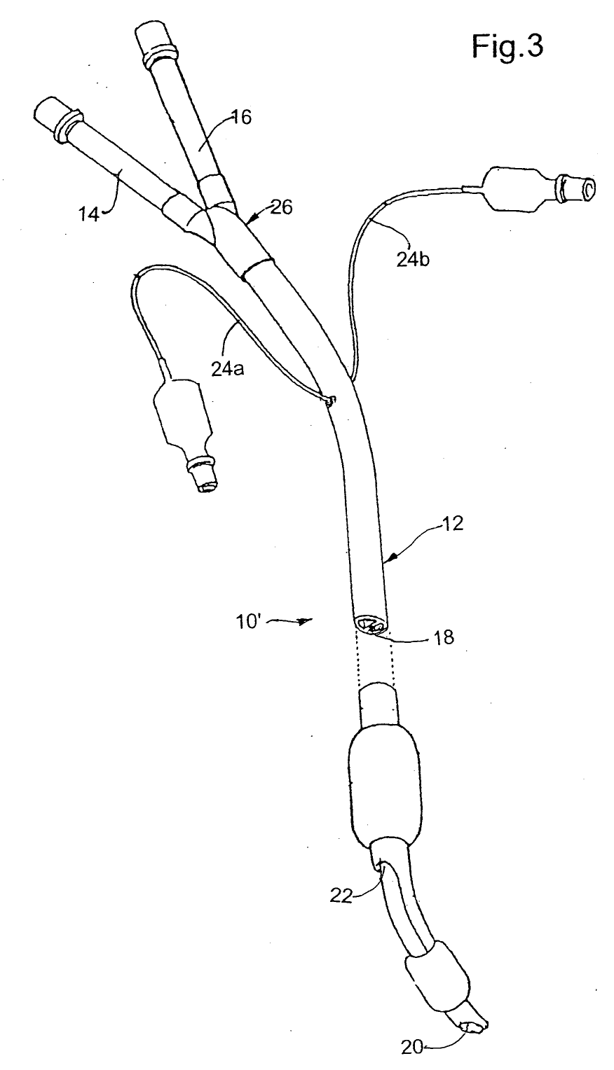

FIG. 3 shows an endotracheal tube manufactured using a connecting piece according to the invention consisting of a double-lumen tube and two attached single-lumen tubes, the endotracheal tube otherwise corresponding to the tube shown in FIG. 1;

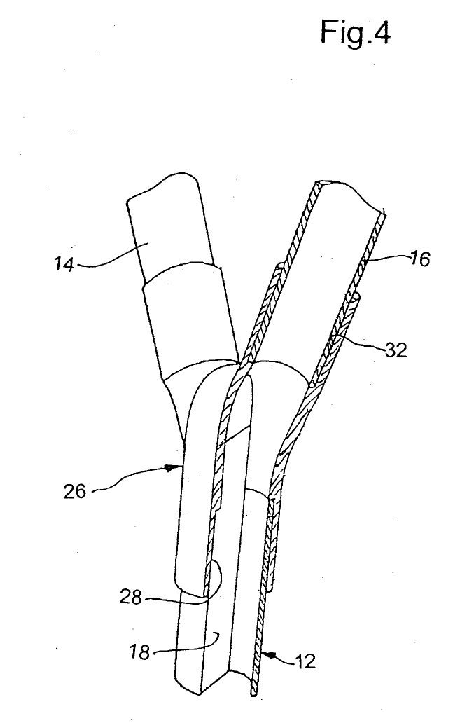

FIG. 4 shows the region of the connection of the double-lumen tube to the attached single-lumen tubes which lies within the chain-dotted area 4 in FIG. 3, wherein the right-hand half of the double-lumen tube, the adjoining region of the connecting piece and the single-lumen tube going off to the right are shown cut away;

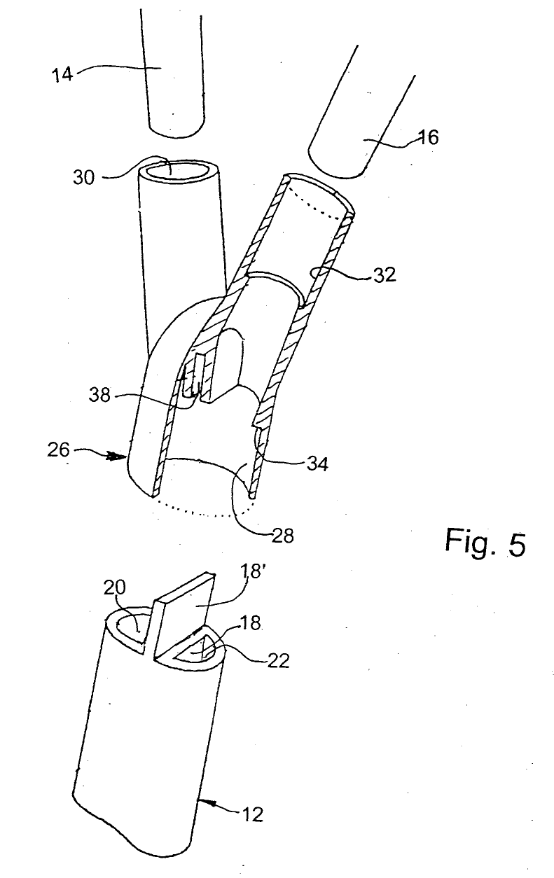

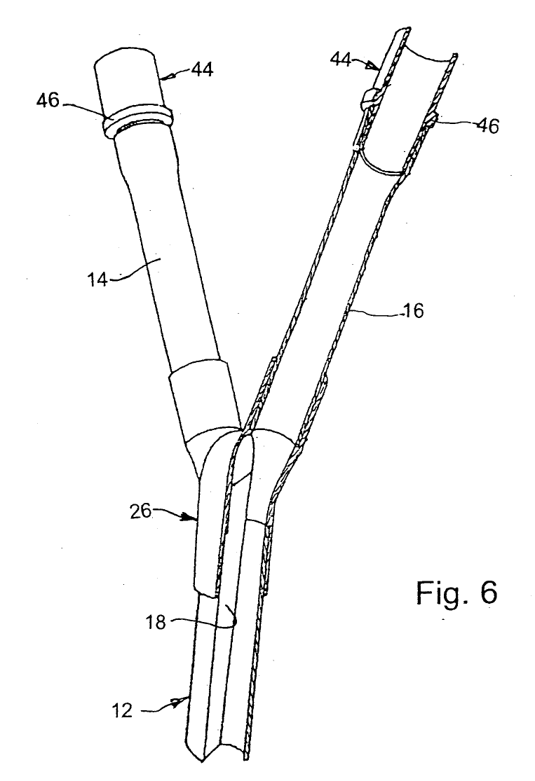

FIG. 5 shows an exploded representation of the connection region 4, wherein the connecting piece is shown partially cut away; and

FIG. 6 shows a view corresponding to FIG. 4 in which connecting stubs are additionally provided on short connecting portions of the single-lumen tubes branching out of the connecting piece, and extensions of the single-lumen tubes adapted in length to the particular examination problem can be attached to these connecting stubs.

A tube 10 according to the prior art which is provided for use in tracheobronchial systems is shown in FIG. 1, this tube consisting of a double-lumen tube 12 and two single-lumen tubes 14 and 16 branching of from the proximal end thereof. The double-lumen tube 12 is divided by a partition 18 into two lumen sections 20 and 22 of different lengths which are separated from one another. Both sections extend from the proximal end region of the double-lumen tube 12 in which two separate inner pipe or tube sections 14a, 16a respectively are inserted into the sections 20, 22 respectively of the double-lumen tube 12 which are separated from one another. One section extends from the distal end region into the trachea and the other extends further into the (right or left) main bronchus. The separate inner pipe or tube sections 14a are introduced with their proximal end sections into the single-lumen tubes 14 and 16 so as to fit. The proximal end of the single-lumen tube 14 is connected by way of the tube section 14a to the section 20 of the double-lumen tube 12. In the same way the single-lumen tube 16 is connected by way of the tube section 16a to the distal end of the section 22.

The tube 10 has two securing and closing balloons, of which one is associated with the section guided into the trachea and the other is associated with the section guided into the bronchus, and these balloons can be inflated by means of two separate pump valves 24a and 24b. These are used to regulate the passage of air, gas and medical or diagnostic and therapeutic instruments, each of these being used depending upon the medical or surgical treatment to be carried out, thus enabling a suitable choice of the pleuropulmonary region to be treated.

The described known construction has the disadvantage that the single-lumen tubes 14 and 16 are mounted on the outer faces of the tube sections 14a, 16a. As a consequence of this a narrowing of the cross-section is produced within the lumina in the connection region, leading to turbulence in the flow of air and considerably impeding the introduction and advance of a bronchoscope, so that the diagnostic and treatment techniques by means of fibre-optic bronchoscopy, which have become increasingly important recently, are made more difficult. Furthermore, there is a risk of damage to the movable tip of the bronchoscope as the extremely thin sheath of the fibre optics is destroyed on the sharp edge of the narrow point.

FIG. 3 shows, in a representation corresponding to FIG. 1, a tube 10′ which can be used in tracheobronchial systems and consists of a double-lumen tube 12 with two single-lumen tubes 14 and 16 branching off from the proximal end thereof, but in this case in an embodiment of the invention the connection of the double-lumen tube 12 to the single-lumen tubes 14 and 16 takes place by way of a connecting piece 26 which is constructed so that the described disadvantages of the known tube 10 are avoided. There is no need for a more detailed description of the tube 10′ because—as far as the connecting piece 26—it corresponds to the known tube 10 described in connection with FIGS. 1 and 2 and components of the two tubes 10 and 10′ which are functionally the same have been allocated the same reference numerals in FIG. 3 as were used in FIG. 1. It is therefore sufficient to given an explanation below with reference to FIGS. 4 to 6 of the differences between the two tubes 10 and 10′ which relate to the special construction of the connecting piece 26.

The connecting piece 26 has the external structure of a Y in which an input connection 28 for the double-lumen tube 12 branches internally into two output connections 30, 32 for the single-lumen tubes 14 and 16.

In order to ensure a substantially step-free continuous transition of the tubes 12 and 14, 16 respectively to be attached to the connecting piece 26, the clear internal cross-section of the input connection 28 intended for connection to the double-lumen tube is substantially complementary in shape to the outer wall of the double-lumen tube 12 in the region where it is attached to the connecting piece 26, this complementary region having in the interior of the connecting piece a narrowing of the cross-section which forms a step abutment 34. The narrowing of the cross-section is dimensioned so that the remaining passage cross-section corresponds exactly to the passage cross-section of the double-lumen tube 12, so that therefore the lumen sections 20, 22 respectively branch smoothly into the output connections 30, 32 for the single-lumen tubes 14, 16. In order for the lumina 20 and 22 of the double-lumen tube which are connected to one another to be sealed of from one another within the connecting piece 26, the partition 18 has an extended portion 18′, that is to say a portion which projects somewhat over the end face of the double-lumen tube 12, which during assembly is inserted into an associated slot 38 in the connecting piece 26 so as to fit.

Also the output connections 30 and 32 are dimensioned in the region where they are connected to the single-lumen tubes 14 and 16 to correspond to the external diameter of these single-lumen tubes, and they extend into the interior of the connecting piece as far as a respective circumferential step-like abutment 42 which determines the depth of insertion of the single-lumen tubes into the respective output connection 30, 32. In this case again the clear passage cross-section of the connecting piece in the region situated before the abutment 42 is chosen so that the passage cross-section of the connecting piece 26 corresponds substantially to the cross-section of the associated internal lumen of the single-lumen tube 14, 16 respectively to be attached. Again, here too a step-like constriction of the passages is avoided in the region where the single-lumen tubes 14, 16 are attached.

In FIG. 6, which otherwise substantially corresponds to FIG. 4, the possibility is also shown of a step-free extension of the proximal ends of the single-lumen tubes by a connecting stub 44 in each case which consists in practice of a short pipe section, the clear internal diameter of which is substantially the same as the clear internal diameter of the single-lumen tubes 14, 16 respectively. An external annular projection 46 running round the circumference constitutes an abutment for the proximal end faces of the single-lumen tubes which are widened resiliently on insertion of the connecting stubs into the insertion region of the connecting stubs 44. Thus the clear internal diameter of the connecting stubs corresponds to the clear internal diameter of the respective associated single-lumen tube in the condition where it is not resiliently widened. An extension of the associated single-lumen tube 14 and/or 16 can then be pushed onto the respective free end region of the connecting stubs 44.

It can be seen that, within the scope of the idea on which the invention is based, variants and further developments of the described embodiment can be provided, as are set out for example in claims 9 to 10 with regard to the material used and in claims 11 to 13 with regard to the manner of connecting the tubes to the connecting stubs 26.

What is essential in any case is that the connection of the lumen sections 20, 22 of the double-lumen tube 12 to the lumina of the single-lumen tubes 14, 16 which branch off from it takes place by means of the connecting piece 26 in such a way that at no point in the passage in the connecting piece 26 is there a constricting step which leads to the stated disadvantages.

1. Connecting piece (16) for a double-lumen tube (12) with two single-lumen tubes (14; 16), particularly for use in the tracheobronchial system, characterised in that the connecting piece (26) has an input connection (28) for the double-lumen tube (12) branching into two output connections (30; 32) for the single-lumen tubes (14; 16), and that the clear passages for the double-lumen tube (12) on the one hand and the output connections for the single-lumen tubes (14; 16) on the other hand are constructed in such a way that in their cross-sectional shape they ensure a substantially step-free continuous transition into the lumina of the tubes to be attached.

2. Connecting piece as claimed in claim 1, characterised in that it has the shape of a Y in which a lumen section extending within the input connection (28) for the double-lumen tube (12) branches into two output lumen sections co-ordinated with the output connections (30; 32) for the single-lumen tubes (14; 16).

3. Connecting piece as claimed in claim 1 or 2, characterised in that the clear internal cross-section of the input connection (28) intended for connection to the double-lumen tube (12) is of substantially complementary shape to the outer wall of the double-lumen tube (12) in the region where it is attached to the connecting piece (26).

4. Connecting piece as claimed in claim 3, characterised in that the internal cross-section of the input connection (28) is provided in the region, opposite the end edge of the double-lumen tube (12) in the proper connection position, of a narrowing of the cross-section forming a circumferential step abutment (34) which is constructed in such a way that the remaining passage cross-section corresponds exactly to the passage cross-section of the double-lumen tube (12).

5. Connecting piece as claimed in one of claims 1 to 4, in which the internal passage cross-section of the double-lumen tube (12) is divided by a continuous central web (18) into two lumina (20; 22) which are approximately semicircular in cross-section, characterised in that in the transition region between the connection for the double-lumen tube (12) and the connections for the single-lumen tubes (14; 16) a slot-like recess (38) is provided in the branching region, whereby in the proper connection position an extended portion (18′) of the web (18) of the double-lumen tube (12) engages and fits in the said recess.

6. Connecting piece as claimed in one of claims 1 to 5, characterised in that the output connections (30; 32) for the single-lumen tubes (14; 16) have in the region where they are attached to the tubes a clear passage cross-section which is complementary to the outer wall of the single-lumen tubes (14; 16) in the connection region.

7. Connecting piece as claimed in claim 6, characterised in that the output connections (30; 32) for the single-lumen tubes (14; 16) each have a circumferential step-like abutment (42) to limit the depth of insertion of the single-lumen tubes, and that the clear passage cross-section of the connecting piece (26) in the abutment region corresponds substantially to the cross-section of the associated lumen of the single-lumen tube (14; 16) to be attached.

8. Connecting piece as claimed in one of claims 1 to 7, characterised in that it is made from a thermoplastic material.

9. Connecting piece as claimed in one of claims 1 to 7, characterised in that it is made from an elastomeric material.

10. Connecting piece as claimed in one of claims 1 to 7, characterised in that it is made from metal.

11. Connecting piece as claimed in one of claims 1 to 5, characterised in that the single-lumen tubes (14; 16) at the output end are produced as an integral die-cast or injection-moulded part with the connecting piece (26).

12. Connecting piece as claimed in one of claims 1 to 11, characterised in that the connection of the regions (28; 30; 32) where the connecting piece (26) is attached to the tubes (12; 14; 16) which are to be attached is constructed as a glued connection.

13. Connecting piece as claimed in one of claims 1 to 9 and 11, characterised in that the connecting piece (26) is injection-moulded directly onto the end portions of the tubes (12; 14; 16) to be attached.
